(19) United States
(12) Patent Application Publication
(10) Pub. No.: US 2013/0206626 A1
(43) Pub. Date: Aug. 15, 2013

(54) METHOD AND DEVICE FOR FABRICATING A PATIENT-SPECIFIC IMPLANT

(71) Applicants: Ralf Schindel, St. Gallen (CH); Reto Artusi, Uhwiesen (CH); Ronald Bauer, Teufen (CH)

(72) Inventors: Ralf Schindel, St. Gallen (CH); Reto Artusi, Uhwiesen (CH); Ronald Bauer, Teufen (CH)

(21) Appl. No.: 13/579,723

(22) PCT Filed: Feb. 19, 2011
(86) PCT No.: PCT/IB2011/000330

§ 371 Date: Apr. 26, 2013

Related U.S. Application Data
(60) Provisional application No. 61/305,973, filed on Feb. 19, 2010.

(51) Int. Cl. A61F 2/28

(52) U.S. Cl. CPC A61F 2/28
USPC 206/438; 53/425

(57) ABSTRACT

The present invention provides a kit, a method of fabrication of the kit and a method of use of the kit for facilitating the fabrication of a patient-specific implant such as a cranial implant. In a preferred embodiment, the kit 30 includes a thermo-formed plastic sheet 32, a portion 34 of which is formed of PP plastics to the three dimensional contour of a bone structure of a specific patient. The thermo-formed sheet 32 optionally doubles as a container 36 for the remaining elements of the kit, which include bone cement 40a, 40b (typically in two part form, each part contained in a separate container device), and, optionally, disposable forming and/or mixing aids 42 such as a spatula. A kneading recess 44 is also formed in the thermoformed sheet 32 of the kit 30. A sheet 46 is adhered via adhesive 48 and extends between flanges 50 of the thermo-formed sheet, to seal and keep sterilized the components 40a, 40b and 42 and the mould recess (shaping form) 34 of the kit 30 within the container 36.

This invention relates to a device in the form of a kit, a method of making the kit, and a method of use of the kit for the fabrication of a patient-specific implant, particularly a hard implant such as a bone implant of a skull fragment, a hip or other bone fragment.

### BACKGROUND OF THE INVENTION

Referring to FIGS. 1A and 1B, in typical neurosurgical operations on the brain, a cranial calotte or calvarial fragment 10 must be removed, in other words, a portion of the skull 12 will be removed, creating an opening 14, to provide access to a ventricle of the skull. In order to close this opening after the operation, it is not possible to simply re-insert the removed fragment 12, largely because swelling of the brain 10 occurs usually after brain surgery and such fragment, if reinserted, would cause unacceptable pressure on the brain. Additionally the geometry of the removed fragment is smaller than the opening in the size of the milling cleft. Therefore, the scalp is closed without reinserting the removed fragment 12 and the patient's head is often protected with an external, removable prosthesis (not shown).

Referring now to FIG. 2, according to the state of the art practiced today, the standard procedure is to wait from two to six months after the operation before inserting a hand-formed, patient-specific prosthesis 16 under the scalp or skin which will close the opening made during surgery. This prosthesis 16 is generally formed during the operation to repair the opening from a two component bone cement such as PMMA (Polymethylmethacrylate), which is kneaded and hand-formed directly on the dura mater 18, the outer soft tissue covering of the brain. Then it must be allowed to harden for between ten and twenty minutes.

The disadvantage of this prior art method is that the time required to fabricate the replacement calotte or fragment 16 together with the set time of the bone cement (during which fabrication the surgeon is not directly focused on the patient but rather on the creation of the prosthesis), the surgeons, nurses and staff are occupied, and the patient has an open wound in his head, and is thus susceptible to infection.

A further disadvantage is that corrections to the geometric form of the replacement fragment 16 are difficult to make, sometimes requiring that the surgeon and his staff restart the process of fabrication of the replacement fragment again, thus further delaying the operation, costing more time and thus money, and occupying surgical infrastructure. The quality of the replacement fragment 16 can depend on the level of experience and fatigue of the surgeon, thus adding risks to the surgical procedure.

A further disadvantage is that to receive the correct and fitting shape of the fragment the surgeon should be able to knead the bone cement with pressure against the bone and dura mater tissue. But giving pressure on the dura mater is allowed only very limited.

Referring now to FIG. 3, a further prior art method for the fabrication of a replacement fragment 20 is a pre-operative method which uses computer tomography data (CT/MRI-data). With this data, a suitable patient-specific replacement fragment 20 can be engineered and direct milled out of titanium or PEEK (an advanced plastics material, Polyetheretherketone). This process is only used where very complex bone geometries must be replaced, because of the costs associated with the fabrication of expensive geometries using this process, as well as the high material costs associated with the use of PEEK plastics approved for medical uses. Further, there is practically no opportunity to modify the geometry intra operatively, given by the toughness and hardness of the material used. Thus, this prosthesis 20 must fit or the operation is aborted and has to be rescheduled for another attempt.

What is needed is a method which avoids the significant disadvantages of the prior art solutions, and to provide the surgeon with a means by which he or she can quickly and inexpensively fabricate a patient-specific replacement calotte fragment 16, thereby saving time and resources, and subjecting the patient to less risk of infection or other complications that may be caused by a longer surgical procedure.

### SUMMARY OF THE INVENTION

An object of the invention is to minimize the time required to perform a surgical intervention, particularly the replacement of a three dimensional hard body part fragment, such as a bone fragment, with a prosthesis.

Another object of the invention is to minimize distraction of a surgeon's attention from the patient while the surgeon is intra-operatively forming a replacement fragment, such as a cranial fragment, and to minimize or eliminate the down-time associated with hardening or setting of the bone cement from which the replacement fragment is formed.

Another object of the invention is to minimize the time and costs associated with a neurosurgical operation.

A kit, a method of fabrication of the kit and a method of use of the kit for facilitating the fabrication of a patient-specific cranial implant is provided which meets the needs identified above.

According to a first aspect of the present invention, there is provided a kit comprising a base body which has a sterilized mould recess with a patient-specific three-dimensional negative surface contour of at least a part of a patient-specific body part structure such as bone, cartilage or teeth, for receiving and shaping a not yet cross-linked and/or not yet hardened kneadable mass; and a removable sealing element hermetically sealing said sterilized mould recess.

This base body with its mould recess enables the surgeon to hand-shape the patent-specific implant by pressing the not yet cross-linked and/or not yet hardened kneadable mass against the mould surface of the mould recess. This guarantees that, one the one hand, at least a first part of the surface of the implant, i.e. the implant surface part interfacing or interacting with the implant recipient's body, is shaped according to the stored patient-specific 3D data, and that, on the other hand, a second part of the surface of the implant, i.e. the implant surface part not interfacing or interacting with the implant recipient's body, will be shaped according the surgeon's hand kneading and/or finger kneading of the not yet cross-linked and/or not yet hardened mass, thus allowing the surgeon to account for practical surgical requirements.

It should be noted that the present invention allows the implant to be made from any biologically acceptable kneadable material which after kneading and shaping will cross-link and/or harden. Depending on the material, such cross-linking and/or hardening will result in an implantable material with mechanical properties ranging from rubber-like to bone-like or even stone-like.

Preferably, the kit's base body has a sterilized kneading recess for receiving said not yet hardened and/or cross-linked mass to be kneaded; and a removable sealing element hermetically sealing said sterilized kneading recess.

This allows the base body to be used both for kneading in the kneading recess and for shaping in the mould recess.

It should be noted that, with some implant materials, it is possible and appreciated by surgeons that the kneaded and shaped implant material may be removed from the mould recess before complete hardening. This may facilitate fitting the implant to the implant recipient's body.

The base body may have a sterilized storage recess in which a first sterilized kneadable mass component and a second sterilized kneadable mass component for preparing said kneadable mass are provided separately from each other; and a removable sealing element hermetically sealing said sterilized storage recess. Preferably, the base body also has a sterilized storage recess in which a sterilized mixing and/or kneading tool for mixing and/or kneading said not yet cross-linked or not yet hardened mass is provided; and a sealing element hermetically sealing said sterilized storage recess.

This enables all the steps of preparing the mass (mixing, kneading) and shaping the mass in the mould recess to be performed on the base body in a sterilized environment.

In a particularly preferred embodiment of the kit, the base body is a thermoformed sheet.

A sealing element may be a foil removably attached to said base body and hermetically sealing the recesses of said base body, or it may be a hermetically sealed bag enclosing said base body.

Advantageously, at least the sterilized surface inside said mould recess and preferably inside said kneading recess is made of an apolar polymer, or said thermoformed sheet is made of an apolar polymer. Such apolar polymer is preferably a C and H based polymer, typically an olefinic or an aromatic polymer, such as polypropylene (PP), polyethylene (PE) or polystyrene (PS).

This prevents unwanted chemical interaction (“sticking”) between the mould surface and some preferred and commonly used kneadable masses such as polymethylmethacrylaLe (PMMA).

According to a second aspect of the present invention, there is provided a method of fabricating a kit as defined in any one of the preceding paragraphs, comprising the following steps:

a) three-dimensionally scanning a patient's body part structure which is to be at least partially replaced and storing said tomographical data;
b) manufacturing a base body having at least one recess with a patient-specific three-dimensional negative surface contour based on said tomographical data;
c) sterilizing the at least one recess of said base body and hermetically sealing it with a removable sealing element.

In a first preferred embodiment of the method of fabricating the kit, step b) consists of manufacturing said base body using an additive (layer-by-layer) process such as laser sintering and providing said base body as the base body of the kit.

In a second preferred embodiment of the method of fabricating the kit, step b) comprises a step b1) for manufacturing an intermediate body using an additive (layer-by-layer) process such as laser sintering; and a step b2) for using said intermediate body as a mould in a thermoforming process such as deep-drawing for thermoforming a sheet material into a thermoformed sheet and providing said thermoformed sheet as the base body of the kit.

According to a third aspect of the present invention, there is provided a method of using a kit as defined in any one of the preceding paragraphs, comprising the following steps:
removing the sealing element from the base body in a sterilized environment, thus exposing the sterilized mould recess;
preparing a kneadable and cross-linkable and/or hardenable (settable) mass;
placing the prepared mass into the sterilized mould recess, thus shaping at least a part of the surface of said mass in accordance with a patient-specific body part structure;
allowing the shaped mass to at least partially cross-link and/or harden; and
removing the at least partially cross-linked and/or hardened shaped mass from said recess.

The mould may be used several times, provided that all the handling takes place in a sterile environment. Also, the surgeon may remove the implant mass before it has completely hardened. This allows the surgeon to make minor adaptations to the shape of the implant mass before or during the fitting of the mass to the implant recipent's body.

Preferably, the kit includes a thermo-formed plastic sheet, formed as a blister pack, to the three dimensional contour of a bone structure of a specific patient typically formed of PP (Polypropylene) plastic (although any other suitable plastic, such as PC, could be used). The same thermo-formed sheet optionally doubles as a container for the remaining elements of the kit, which include bone cement (see http://en.wikipedia.org/wiki/Bone_cement, the contents of which are incorporated herein by reference, referring to bone cement available in two part form, each part contained in a separate container tube), and, optionally, disposable forming aids such as a spatula or a mixing cup. A sealing sheet may be adhered to and extended between flanges of the thermo-formed sheet, to seal the components of the kit within the kit container.

Preferably, the main kit is a thermo-formed sealed packed sterilized plastic sheet which contains the engineered negative form of the patient-specific calotte (FIGS. 8A and 8B).

This kit could be enlarged or added by a patient-specific model of the wound or bone contour to control geometry of the set PMMA implant.

Preferably, the method of fabrication of the kit includes several steps. In a first fabrication step, CT/MRI data of a patient is created using a CT or MRI scanner. In a second fabrication step, the patient-specific skull fragment is engineered from CT/MRI data, including inputs and modifications requested by the surgeon (e.g. consideration of muscle placement which may change the geometry of the fragment, etc). In a third fabrication step, the deep draw form is engineered through use of 3D data of the form of the patient-specific fragment. Optionally, recesses for accommodation of standard containers of bone cement, a spatula or mixer, are included in the data so as to create a blister pack kit including all components and tools needed to complete the operation. In a fourth fabrication step, the custom mould to thermo form the plastic blister is fabricated by, for example, laser sintering. Optionally, this mould is made as an insert to a thermoforming mold which includes a form for creating the above-mentioned recesses for accommodation of standard containers of bone cement, a spatula or mixer. In a fifth fabrication step, the kneading form is fabricated through, e.g., thermoforming a PP sheet, optionally, together with additional compartments for other components needed to complete the operation. In a sixth fabrication step, the kneading form is optionally filled with the additional components, and at least selectively sterilized by sterilizing and sealing the PP kneading form (partial or one-sided mould).

Preferably, the method of use of the kit includes several steps. In a first step, the kit is opened, exposing the patient-specific thermoformed form. In a second step, the two components of the bone cement (PMMA) are kneaded together to the desired consistency. In a third step, the kneaded bone cement is placed over the patient-specific thermoformed form in order to form the implant. In a fourth step, the bone cement is allowed to set. In a fifth step, the formed implant is placed on the skull of the patient in a position where it will be fixed. Note that to aid in positioning where the hole is a circular hole formed with core drill bits, a notch, formed on the hole, may be mirrored in the patient-specific PMMA implant. In a sixth step, the surgeon checks if the implant properly fits in place. If not, in a seventh step, the surgeon evaluates whether to attempt to modify the replacement fragment. In an eighth step, if the surgeon cannot make satisfactory modifications, then the method returns to the first step, and a new fragment is made. In a ninth step, the implant is fixed in place, thus closing the wound.

Note that if there is a problem with the fit, the surgeon evaluates whether to attempt to modify the replacement fragment. If the surgeon cannot make satisfactory modifications, then the method is reinitiated, with a new implant being made using the same mould (taking advantage of the fact that the mould is reusable), and returns to the first step, and a new fragment is made and subsequently fixed in place.

### BRIEF DESCRIPTION OF THE DRAWINGS

FIGS. 1A and 1B are perspective views of a skull and in particular, the opening and patient-specific implant whose fabrication is the object of the invention.

FIG. 2 is a collage of views showing steps of a method of the prior art.

FIG. 3 is a perspective view of an implant made according to an alternate method of the prior art.

FIG. 4A is a top view of a kit of the invention.

FIG. 4B is a cross sectional view of a kit of the invention, taken along line A-A of FIG. 4A.

FIG. 5A is a flow chart of the method of fabrication of the kit of the invention.

FIG. 5B is a schematic view of CT/MRI data capture of a patient's skull.

FIG. 5C is a perspective view of a custom (i.e., patient-specific) deep draw form (mould) fabricated by, for example, laser sintering.

FIG. 5D is a schematic view of a laser sintering process practiced by Inspire AG, Switzerland.

FIG. 5E in a schematic diagram of a typical thermoforming process used to shape the PP form (one-sided mould) of the invention.

FIGS. 6A and 6B are perspective views of typical thermoformed PP forms (one-sided moulds).

FIG. 7 is a flow chart of the method of use of the kit of the invention.

FIGS. 8A and 8B are top and cross-sectional side views of the thermoformed form/mould of the invention.

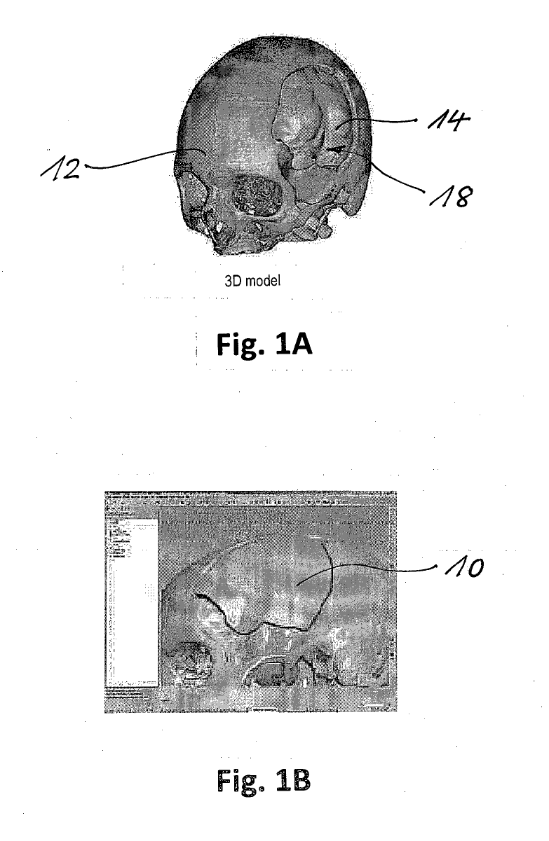

Those skilled in the art will appreciate that elements in the Figures are illustrated for simplicity and clarity and have not necessarily been drawn to scale. For example, dimensions may be exaggerated relative to other elements to help improve understanding of the invention and its embodiments. Furthermore, when the terms ‘first’, ‘second’, and the like are used herein, their use is intended for distinguishing between similar elements and not necessarily for describing a sequential or chronological order. Those skilled in the art will therefore understand that such terms may be interchangeable with other terms, and that the embodiments described herein are capable of operating in other orientations than those explicitly illustrated or described.

### DETAILED DESCRIPTION OF THE PREFERRED EMBODIMENT

The following description is not intended to limit the scope of the invention in any way as they are exemplary in nature and serve to describe the best mode of the invention known the inventors as of the filing date hereof. Consequently, changes may be made in the arrangement and/or function of any of the elements described in the disclosed exemplary embodiments without departing from the spirit and scope of the invention.

In the instant application, a kit facilitating an implant operation, a method of fabrication of the kit and a method of use of the kit for facilitating the fabrication of a patient-specific cranial implant is provided which reduces the time required for preparation of such implant, increases the focus of the surgeon on the patient and minimizes the time which the patient is exposed to possible infection. The kit provides a means for pre-operative preparation of the implant, thereby eliminating intra-operative preparation and, consequently, eliminating the wait time associated with the setting of the bone cement.

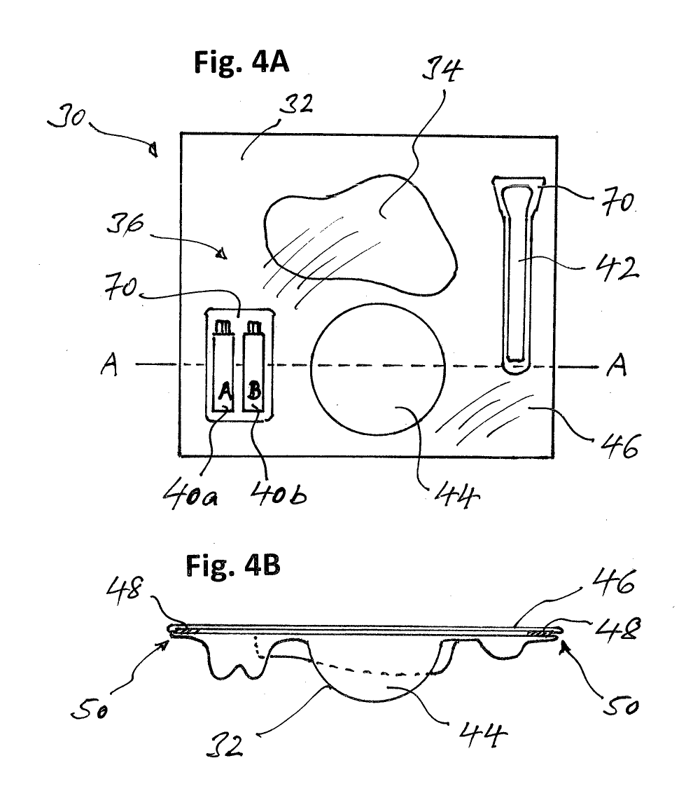

Referring now to FIGS. 4A and 4B, the kit 30 includes a thermo-formed plastic sheet 32, a portion 34 of which is formed (typically of PP plastics) to the three dimensional contour of a bone structure of a specific patient. The thermo-formed sheet 32 optionally doubles as a container 36 for the remaining elements of the kit, which include bone cement 40a, 40b (typically in two part form, each part contained in a separate container device), and, optionally, disposable forming and/or mixing aids 42 such as a spatula. A kneading recess 44 may also be formed in the thermoformed sheet 32 of the kit 30. A sheet 46 may be adhered to (e.g., via adhesive 48) and extends between flanges 50 of the thermo-formed sheet, to seal and keep sterilized the components 40a, 40b and 42 and the mould recess or shaping form 34 of the kit 30 within the container 36.

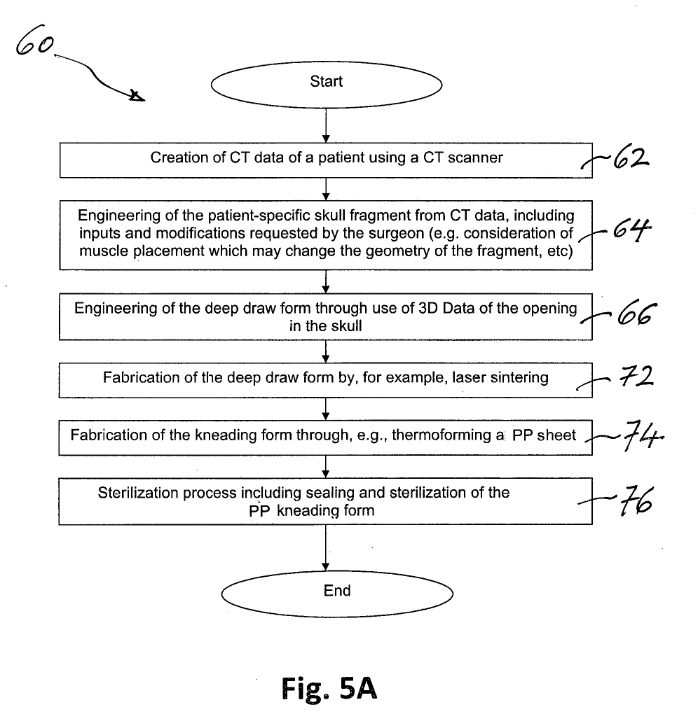

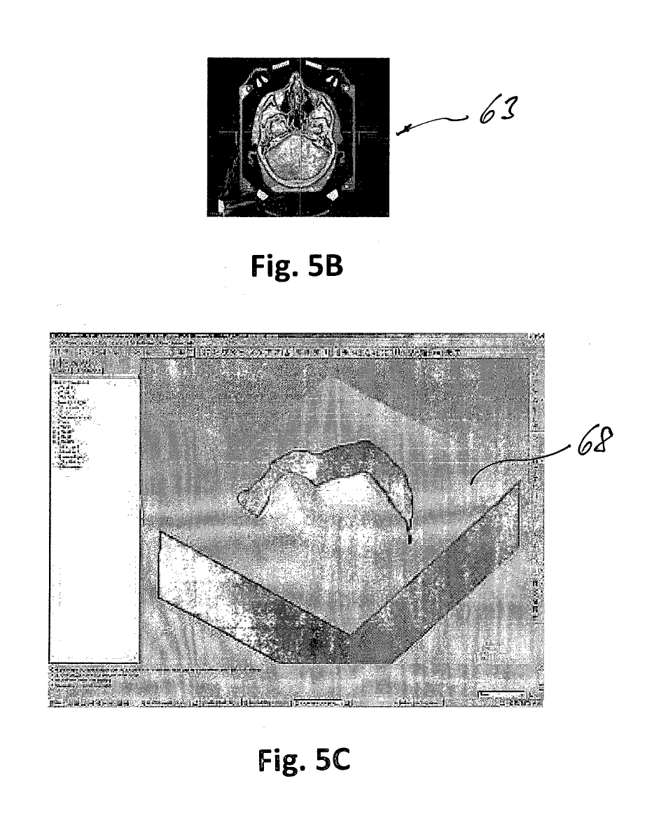

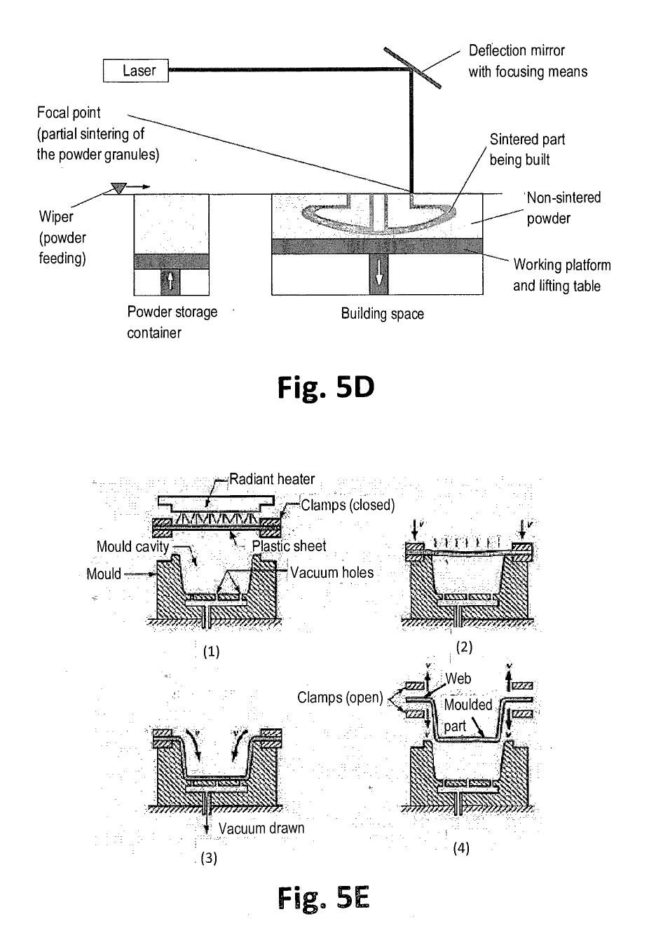

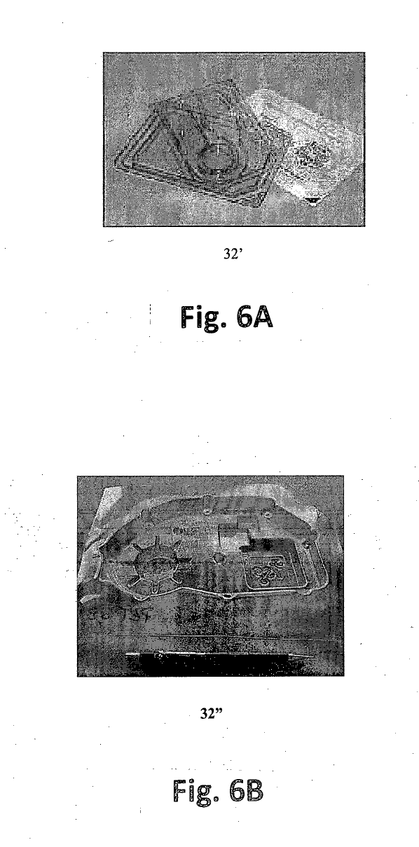

Referring now to FIGS. 5A to 5E, the method 60 of fabrication of the kit 30 includes several steps. Referring in particular to FIG. 5B, in a first fabrication step 62, CT/MRI data 63 of a patient is created using a CT or MRI scanner. In a second fabrication step 64, the form of the patient-specific skull fragment is engineered from CT/MRI data, including inputs and modifications requested by the surgeon (e.g. consideration of muscle placement which may change the geometry of the fragment, etc). In a third fabrication step 66, the deep draw form 68 is designed using the 3D data 63 of the form of the patient-specific skull fragment 10. Optionally recesses 70 for accommodation of standard containers of bone cement 40a, 40b, a spatula or mixer 42, are included in the data so as to create a blister pack kit 30 including all operation-specific components and tools needed to complete the operation. Referring to FIG. 5C, in a fourth fabrication step 72, the custom deep draw form 68 is fabricated by, for example, laser sintering, described in FIG. 5D using methods known in the art and/or practiced by Inspire AG, Switzerland. Optionally, this form 68 is made as an insert to a thermoforming mold which includes a form for creating the above-mentioned recesses 70. Referring now to FIG. 5E, in a fifth fabrication step 74, the kneading form 32 is fabricated through, e.g., thermoforming a PP sheet, optionally, together with additional compartments for other components needed to complete the operation. See FIGS. 6A and 6B for examples of thermoformed sheets 32′ and 32″. In a sixth fabrication step 76, the kneading form 32 is sterilized which comprises a sealing and sterilization of the PP kneading form. Examples of PP forms shaped by thermoforming are provided in FIGS. 6A and 6B.

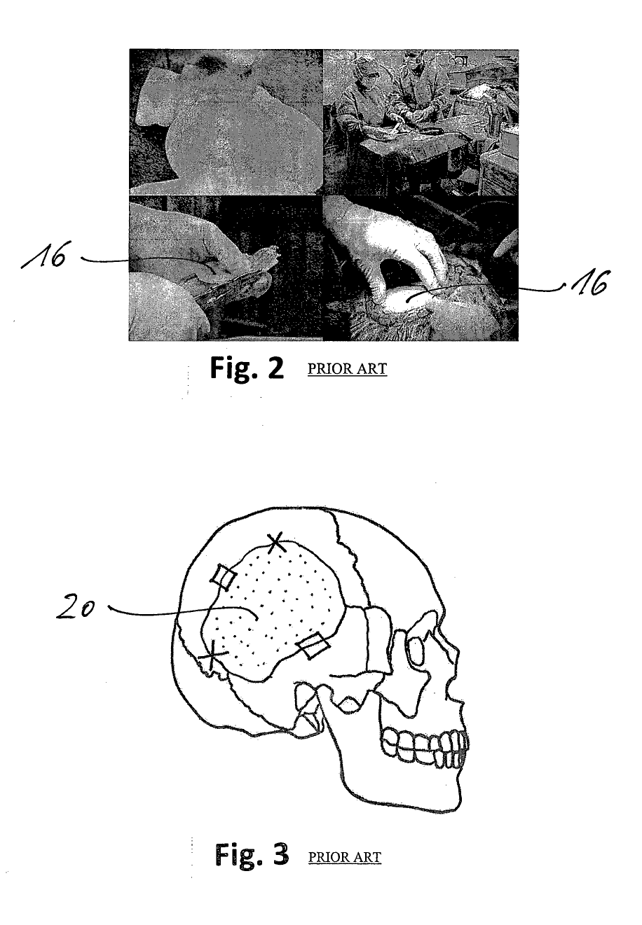

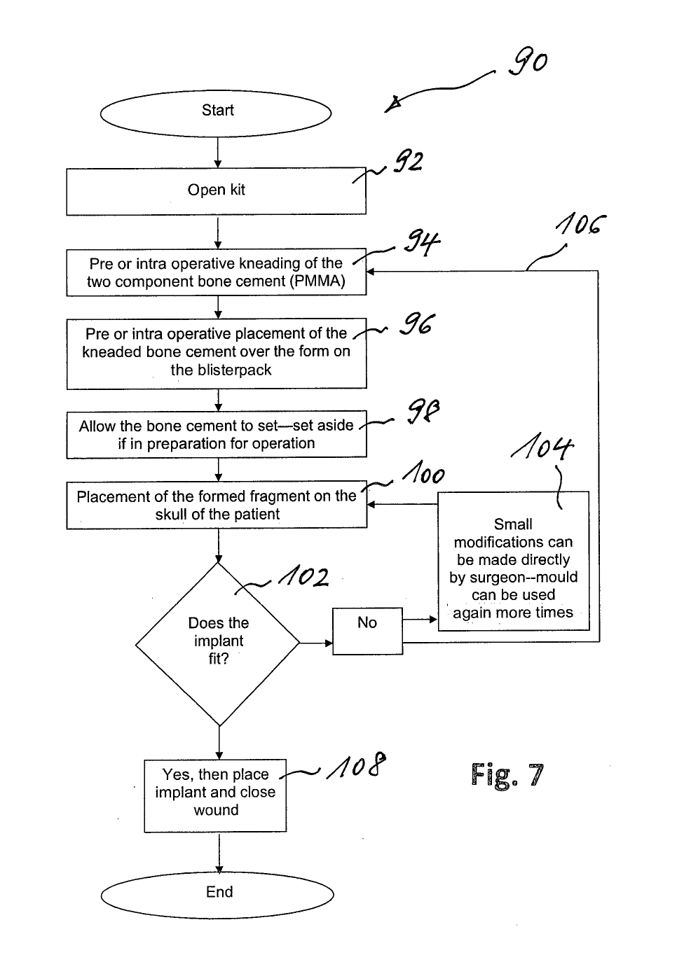

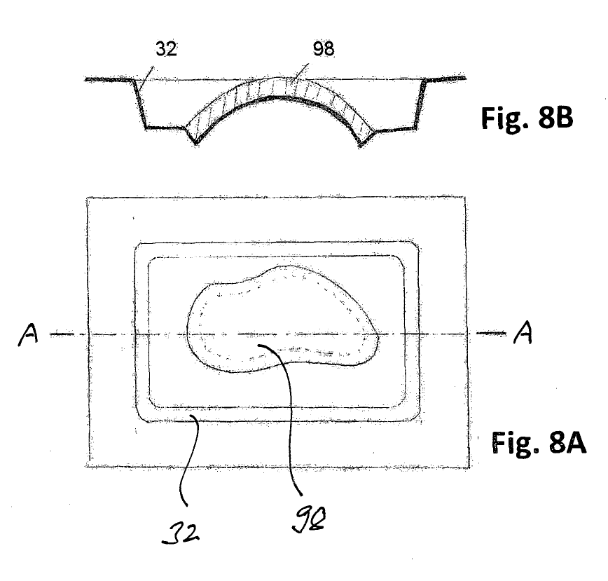

Referring now to FIGS. 7 and 8A and 8B, the method of use 90 of the kit 30 includes several steps. In a first step 92, the kit is opened, exposing the patient-specific thermoformed form. In a second step 94, the two components 40a, 40b of the bone cement (PMMA) are mixed and kneaded to the desired consistency (see FIGS. 2A-2B). In a third step 96, the kneaded bone cement 98 is placed over the patient-specific thermoformed form 32 in order to shape and thus form the implant 16. In a fourth step 98, the kneaded bone cement 98 is allowed to set. In a fifth step 100, the formed implant 16 is placed on the skull 12 of the patient in the only position where it will be fixed (in the pre-surgical case this step can be proved with a laser sintered model of the skull). In a sixth step 102, the surgeon checks if the implant 16 properly fits in place. If not, in a seventh step 104, the surgeon evaluates whether to attempt to modify the replacement fragment. In an eighth step 106, if the surgeon cannot make satisfactory modifications, then the method returns to the first or second step 92, 94, and a new implant 16 is made. In a ninth step 108, the implant 16 is fixed in place, thus closing the wound.

Skilled artisans will appreciate that the configurations depicted in the figures are provided for representative and convenient illustration and that many other configurations may be alternatively, conjunctively and/or sequentially employed to produce substantially the same result. The present invention may be described herein in terms of functional block components and various processing steps. It should be appreciated that such functional blocks may be realized by any number of hardware and/or software components configured to perform the specified functions. For example, the present invention may employ various integrated circuit components, e.g., memory elements, processing elements, logic elements, data structures, and/or the like, which may carry out a variety of functions under the control of one or more microprocessors or other control devices. Similarly, the software elements of the present invention may be implemented with any programming or scripting language and/or any programming or scripting language now known or hereafter derived or otherwise described in the art, with the various algorithms being implemented with any combination of data structures, objects, processes, routines or other programming elements.

It should be appreciated that the particular implementations shown and described herein are representative of the invention and its best mode and are not intended to limit the scope of the present invention in any way. Indeed, for the sake of brevity, conventional data networking, application development and other functional aspects of the systems (and components of the individual operating components of the systems) may not be described in detail herein. Furthermore, the connecting lines shown in the various figures contained herein are intended to represent exemplary functional relationships and/or physical couplings between various elements. It should be noted that many alternative or additional functional relationships or physical connections may be present in a practical system.

As will be appreciated by those skilled in the art, the present invention may be embodied as a method, a system, a device, and/or a computer program product.

Moreover, the system contemplates the use, sale and/or distribution of any goods, services or information having similar functionality described herein.

The specification and figures are to be considered in an illustrative manner, rather than a restrictive one and all modifications described herein are intended to be included within the scope of the invention claimed, even if such is not specifically claimed at the filing of the application. Accordingly, the scope of the invention should be determined by the claims appended hereto or later amended or added, and their legal equivalents rather than by merely the examples described above. For instance, steps recited in any method or process claims may be executed in any order and are not limited to the specific order presented in any claim. Further, the elements and/or components recited in any apparatus claims may be assembled or otherwise operationally configured in a variety of permutations to produce substantially the same result as the present invention. Consequently, the invention is not limited to the specific configuration recited in the claims.

The direct benefits of the invention are reduced OR time, an optimal form of the implant and a correspondingly better fit, the use of tried and proven bone cement which allows for modification using common surgical tools, and a corresponding reduction in healthcare costs.

In an advantage of the invention, the time required to perform a surgical intervention is reduced, particularly the time required for the replacement of a three dimensional bone fragment 10 with a prostheses 16, 20.

In another advantage, the operation can be performed quickly, using bone cement, a material which has been tested in many applications and has been known and trusted for years. Using bone cement, if a correction to the form of the implant 16 is required, this can be done by the surgeon or an assistant using readily available surgical tools. See FIG. 2C.

In another advantage, the distraction of a surgeon's attention from the patient is reduced during the time that the surgeon would otherwise be intra-operatively forming a replacement cranial fragment 16.

In another advantage, the invention minimizes or eliminates the down-time associated with hardening or setting of the bone cement from which the replacement cranial fragment 16 is formed.

In another advantage, the invention minimizes the costs associated with a neurosurgical operation.

In another advantage, the delivery time from receipt of the CT/MRI data is reduced to about one to three weeks.

Benefits, other advantages and solutions mentioned herein are not to be construed as critical, required or essential features or components of any or all the claims.

As used herein, the terms “comprises”, “comprising”, or any variation thereof, are intended to refer to a non-exclusive listing of elements, such that any process, method, article, composition or apparatus of the invention that comprises a list of elements does not include only those elements recited, but may also include other elements described in this specification. The use of the term “consisting” or “consisting of” or “consisting essentially of” is not intended to limit the scope of the invention to the enumerated elements named thereafter, unless otherwise indicated. Other combinations and/or modifications of the above-described elements, materials or structures used in the practice of the present invention may be varied or otherwise adapted by the skilled artisan to other design without departing from the general principles of the invention.

The patents and articles mentioned above are hereby incorporated by reference herein, unless otherwise noted, to the extent that the same are not inconsistent with this disclosure.

Other characteristics and modes of execution of the invention are described in the appended claims.

Further, the invention should be considered as comprising all possible combinations of every feature described in the instant specification, appended claims, and/or drawing figures which may be considered new, inventive and industrially applicable.

The copyrights in any appendix hereto are owned by the Applicant(s) or their assignee and, with respect to express Licensees of the rights defined in one or more claims herein, no implied license is granted herein to use the invention as defined in the remaining claims. Further, vis-à-vis third parties, including the public, no express or implied license is granted to reproduce, prepare derivative works, distribute copies, display, or otherwise use this patent specification, inclusive of the appendix hereto and any computer program comprised therein, except as an appendix to a patent issuing hereon.

Multiple variations and modifications are possible in the embodiments of the invention described here. Although certain illustrative embodiments of the invention have been shown and described here, a wide range of modifications, changes, and substitutions is contemplated in the foregoing disclosure. While the above description contains many specifics, these should not be construed as limitations on the scope of the invention, but rather as exemplifications of one or another preferred embodiment thereof. In some instances, some features of the present invention may be employed without a corresponding use of the other features. Accordingly, it is appropriated that the foregoing description be construed broadly and understood as being given by way of illustration and example only, the spirit and scope of the invention being limited only by the claims which ultimately issue in this application.

1. A kit comprising a base body which has a sterilized mould recess with a patient-specific three-dimensional negative surface contour of at least a part of a patient-specific body part structure such as bone, cartilage or teeth, for receiving and shaping a not yet cross-linked and/or not yet hardened kneadable mass; and a removable sealing element hermetically sealing said sterilized mould recess.

2. The kit according to claim 1, wherein said base body has a sterilized kneading recess for receiving said not yet hardened or cross-linked mass to be kneaded; and a removable sealing element hermetically sealing said sterilized kneading recess.

3. The kit according to claim 1, wherein said base body has a sterilized storage recess in which a first sterilized kneadable mass component and a second sterilized kneadable mass component for preparing said kneadable mass are provided separately from each other; and a removable sealing element hermetically sealing said sterilized storage recess.

4. The kit according to any one of claim 1, wherein said base body has a sterilized storage recess in which a sterilized mixing and/or kneading tool for mixing and/or kneading said not yet cross-linked or not yet hardened mass is provided; and a sealing element hermetically sealing said sterilized storage recess.

5. The kit according to any one of claim 1, wherein said base body is a thermoformed sheet.

6. The kit according to any one of claim 1, wherein a sealing element is a foil removably attached to said base body and hermetically sealing the recesses of said base body.

7. The kit according to any one of claim 1, wherein a sealing element is a hermetically sealed bag enclosing said base body.

8. The kit according to claim 1, wherein at least the sterilized surface inside said mould recess and preferably inside said kneading recess is made of an apolar polymer such as polypropylene (PP), polyethylene (PE) or polystyrene (PS).

9. The kit according to any one of claim 1, wherein said thermoformed sheet is made of an apolar polymer such as polypropylene (PP), polyethylene (PE) or polystyrene (PS).

10. A method of fabricating a kit as defined in claim 1, comprising the following steps:
a) three-dimensionally scanning a patient's body part structure which is to be at least partially replaced and storing said tomographical data;
b) manufacturing a base body having at least one recess with a patient-specific three-dimensional negative surface contour based on said tomographical data;
c) sterilizing the at least one recess of said base body and hermetically sealing it with a removable sealing element.

11. The method according to claim 10, wherein step b) consists of manufacturing said base body using an additive (layer-by-layer) process such as laser sintering and providing said base body as the base body of the kit.

12. The method according to claim 10, wherein step b) comprises a step b1) for manufacturing an intermediate body using an additive (layer-by-layer) process such as laser sintering; and a step b2) for using said intermediate body as a mould in a thermoforming process such as deep-drawing for thermoforming a sheet material into a thermoformed sheet and providing said thermoformed sheet as the base body of the kit.

13. A method of using a kit according to claim 1, comprising the following steps:
removing the sealing element from the base body in a sterilized environment, thus exposing the sterilized mould recess;
preparing a kneadable and cross-linkable and/or hardenable (settable) mass;
placing the prepared mass into the sterilized mould recess, thus shaping at least a part of the surface of said mass in accordance with a patient-specific body part structure;
allowing the shaped mass to at least partially cross-link and/or harden; and
removing the at least partially cross-linked and/or hardened shaped mass from said recess.
